(19) United States
(12) Patent Application Publication
(10) Pub. No.: US 2009/0162401 A1
(43) Pub. Date: Jun. 25, 2009

(54) Streptococcus Iniae Phosphoglucomutase is a Virulence Factor and Target for Vaccine Development

(76) Inventors: John T. Buchanan, San Diego, CA (US); Jason A. Stannard, San Diego, CA (US); Xavier Lauth, San Diego, CA (US); Vaughn E. Ostland, La Quinta, CA (US); Mark E. Westerman, San Diego, CA (US); Victor Nizet, San Diego, CA (US)

Correspondence Address: DLA PIPER LLP (US), 4365 EXECUTIVE DRIVE, SUITE 1100, SAN DIEGO, CA, 92121-2133, US

(21) Appl. No.: 11/884,600

(22) PCT Filed: Feb. 17, 2006
(86) PCT No.: PCT/US06/05655

§ 371 Date: Oct. 7, 2008

Related U.S. Application Data
(60) Provisional application No. 60/654,148, filed on Feb. 18, 2005.
(60) Provisional application No. 60/655,614, filed on Feb. 22, 2005.

(51) Int. Cl. A61K 39/09; C12N 1/20; C12N 1/21; C07H 21/04; C07K 14/315; C12Q 1/68; C40B 40/02

(52) U.S. Cl. 424/244.1; 435/253.4; 435/252.3; 536/23.7; 536/24.33; 530/350; 435/6; 506/14

(57) ABSTRACT

The present invention relates generally to the identification of virulence factors from Streptococcus iniae. Specifically, the present invention relates to the identification, characterization and sequencing of a gene for phosphoglucomutase gene, and to a live attenuated strain of S. iniae deficient in phosphoglucomutase useful as a vaccine in aquatic species, such as fish.

### CROSS REFERENCE TO RELATED APPLICATIONS

This application claims the benefit of priority under 35 U.S.C. § 120 of U.S. Application Ser. Nos. 60/655,614 filed Feb. 22, 2005 and 60/654,148 filed Feb. 18, 2005, the contents of which are incorporated by reference in their entirety.

### STATEMENT OF GOVERNMENT SUPPORT

This invention was in part made with government support under Grant No.: 2002-35204-11623 from USDA CSREES National Research Initiative (VN). The United States government may have certain rights in this invention.

### BACKGROUND OF THE INVENTION

1. Field of the Invention

The present invention relates generally to the identification of virulence factors from marine pathogens and more specifically to methods for producing attenuated strains of S. iniae useful as vaccines and for preventing S. iniae disease.

2. Background

In the United States, aquaculture provides >25% of our seafood supplies and is the fastest growing sector of the agriculture industry. With increased development of intensive aquaculture operations, disease has become a significant hurdle to the profitable culture of fish and shellfish.

One of the most destructive infections is the fatal meningoencephalitis produced by Streptococcus iniae, which causes hundreds of millions of dollars in losses annually. More than 30 species of fish are reportedly susceptible to S. iniae disease, including trout, yellowtail, tilapia, barramundi, and hybrid striped bass (HSB). Occasionally, S. iniae can cause serious zoonotic infections in humans who injure themselves while handling infected fish.

Relatively little is known about the genetics of S. iniae and the pathogenic mechanisms underlying its virulence in fish. Treatment options are few and limited. Large-scale use of antibiotics in aquaculture is costly, impacts the environmental impacts through farm effluent release, and can lead to immunosuppression and drug resistance. Development of an effective vaccine to infection would be of great benefit to the U.S. aquaculture industry and domestic seafood supplies.

Thus, there is a need for methods and vaccine compositions to prevent S. iniae disease.

### SUMMARY OF THE INVENTION

The present invention provides an isolated Streptococcus iniae bacterium comprising a phosphglucolmutase deficiency. The deficiency can be a decrease in phosphglucolmutase enzyme activity, for example, of at least about 10-fold compared to a wild type Streptococcus iniae bacterium. Typically, this isolated bacterium of the invention is more sensitive to immune clearance mechanisms than a wild type Streptococcus iniae bacterium. For example, the isolated bacterium may be more sensitive to antimicrobial peptides, such as moronecidin or mCRAMP.

In certain embodiments of the invention, the isolated Streptococcus iniae has at least one phenotypic difference when compared to a wild type Streptococcus iniae bacterium. The difference can be decreased buoyancy; increased cell hydrophobicity; decreased surface associated exopolysaccharide capsule; decreased binding to cytochrome C; decreased surface negative charge; or increased cell volume.

A deficiency in phosphglucolmutase can result from a mutation in a gene encoding a phosphglucolmutase, such as a deletion or an insertion (e.g. of a transposon).

The mutation can occur in coding sequences (i.e. open reading frames) or in control sequences (e.g., promoters). The deficiency can, for example, be absent production of phosphglucolmutase protein, or production of phosphglucolmutase protein that is avirulent (e.g., in an aquatic species such as a fish).

In one embodiment of the invention, the phosphglucolmutase includes the sequence set forth in SEQ ID NO:6, which can be encoded by a polynucleotide including the sequence set forth in SEQ ID NO: 5, as also provided by the invention.

The present invention further provides vaccines comprising phosphglucolmutase deficient S. iniae bacteria, which can be live or killed. The vaccines of the invention can contain adjuvants, stabilizers and/or diluents. Phosphglucolmutase deficient S. iniae may be formulated with suitable carriers into pharmaceutical or veterinary compositions.

The present invention further provides methods for preventing Streptococcus iniae disease in a subject comprising administering phosphglucolmutase deficient S. iniae to the subject, wherein the subject develops immunity to the bacterium. S. iniae disease can be meningoencephalitis, for example in aquatic species such as fish, for example a hybrid striped bass or tilapia. In other embodiments, the aquatic species can be a channel catfish, rainbow trout, eel, yellowtail, turbot, or sea bass.

According to the methods of the invention, the S. iniae bacteria or vaccines derived therefrom, can be administered intraperitoneally, subcutaneously, intravenously, intramuscularly, orally (e.g., in food) or by immersion.

The present invention also provides isolated phosphglucolmutase polynucleotides e.g., comprising the sequence set forth in SEQ ID NO:5, which can, for example, be contained in a plasmid, such as pSiPGM. In certain embodiments of the invention, primers comprising approximately 15-50 nucleotides of the phosphglucolmutase sequence are also provided.

The invention also provides isolated polypeptides comprising the sequence set forth in SEQ ID NO:6, and immunogenic fragments thereof, both of which can be expressed from an expression vector such as pSiPGM.

Methods for identifying a virulence factor in a marine pathogen, are also encompassed by the invention. The steps of such methods can include: (a) providing a marine pathogen; (b) randomly mutagenizing the pathogen at the rate of one mutation per cell: (c) isolating clones of the randomly mutated pathogens; (d) identifying a mutant of reduced virulence by comparing the virulence of a randomly mutated clone with the unmutagenized pathogen; and (e) determining the nucleotide position of the mutation in the mutant of reduced virulence, thereby identifying a virulence factor in a marine pathogen. According to these methods, the marine pathogen can infect aquatic species, such as fish and particularly hybrid striped bass (HSB), and can be a bacterium, such as Streptococcus iniae.

According to the invention, randomly mutagenizing typically comprises transposon-mediated mutagenesis where the transposon can be, for example, Tn917. A collection of such randomly mutagenized pathogens, e.g., a transposon insertion library comprising a plurality of S. iniae bacteria, is also encompassed by the invention.

In order to identify a mutant of reduced virulence, the following steps can be taken: (i.) infecting a first subject susceptible to the marine pathogen with a randomly mutated version of the pathogen; (ii.) infecting a second subject susceptible to the marine pathogen with the wild-type marine pathogen; and (iii.) comparing the pathogenic response of the first subject and the second subject. A lesser pathogenic response of the first subject compared to the second subject is indicative of reduced virulence of the mutant, and thus identifies a mutant of reduced virulence.

In one embodiment, there is provided an in vivo method for producing about 2-3 orders of magnitude more transmembrane protein in a mammalian cell as compared to standard methods by contacting a nucleic acid sequence encoding the transmembrane protein and operably linked to regulatory elements with a skeletal muscle cell of a subject, and introducing the nucleic acid sequence into the cell using electroporation, wherein expression of the transmembrane protein is by endogenous translation of the nucleic acid sequence, and thereby producing 2-3 orders of magnitude more transmembrane protein in a mammalian cell as compared to standard methods. The method provided can be accomplished by, for example, by optimization of various steps including the contacting and introducing steps.

Also provided by the invention are marine pathogens containing site-directed mutations in a virulence gene. Such mutation can be the result of deletion and/or insertion by recombination, including but not limited to, homologous recombination.

### BRIEF DESCRIPTION OF THE DRAWINGS

FIG. 1A shows the chromosomal location of the S. iniae pgm gene and site of Tn917AE insertion in promotor motifs upstream of translational start site. FIG. 1B shows an alignment of S. iniae PGM amino acid sequences with known bacterial PGMs and indications of putative conserved functional domains; FIG. 1C lists the percent identity and similarity between S. iniae PGM and other streptococcal PGMs.

FIG. 2A is a graph showing the PGM activity in wild type S. iniae and the ΔPGM mutant; FIG. 2B is a graph comparing the PGM activity of wild type E. coli, the PGM deficient E. coli mutant POP458′ and POP458 mutant expressing S. iniae PGM from the recombinant plasmid pSiPGM.

FIG. 3A shows a Kaplan-Meier survival plot of HSB challenged with 4×10⁵ cfu of wild-type S. iniae, ΔPGM mutant or the pSiPGM-complemented ΔPGM; FIG. 3B shows bacterial counts in blood of fish 24 hrs after infection wild-type S. iniae, ΔPGM mutant or the pSiPGM-complemented ΔPGM.

FIG. 4A is a graph showing the survival ratio (mean±SE) in hybrid striped bass (HSB) blood of wild-type Streptococcus iniae and the isogenic ΔPGM mutant; FIGS. 4B and 4C show the kinetics of killing of wild-type S. iniae, ΔPGM mutant or the complemented mutant by the HSB AMP moronecidin (FIG. 4B) and the murine AMP mCRAMP (FIG. 4C).

FIG. 5A depicts the decreased buoyancy of the ΔPGM mutant compared to wild-type S. iniae as measured by enhanced migration through a Percoll gradiant; FIG. 5B depicts the increased hydrophobicity of the ΔPGM mutant compared to wild-type S. iniae as measured by partition into N-hexadecane; FIG. 5C are transmission electron micrographs of wild-type S. iniae and the ΔPGM mutant.

FIG. 6A shows bacterial counts in blood, spleen and brain of hybrid striped bass (HSB) at different time points after intraperitoneal challenge with wild-type S. iniae and the ΔPGM mutant; FIGS. 6B and 6C show the histology of HSB tissues infected with wild-type S. iniae (FIG. 6B) and the ΔPGM mutant (FIG. 6C).

FIG. 7 is a graph showing the 21 day survival of HSB that were vaccinated with various doses of live-attenuated S. iniae ΔPGM and then challenged with a lethal dose of wild-type S. iniae.

### DETAILED DESCRIPTION OF THE INVENTION

The practice of the present invention will employ, unless otherwise indicated, conventional techniques of molecular biology, microbiology, recombinant DNA technology, and immunology, which are within the skill of the art. Such techniques are explained fully in the literature. See, e.g., Sambrook, Fritsch & Maniatis, “Molecular Cloning: A Laboratory Manual, Vols. I, II and III,” Second Edition (1989); Ausubel et al., “Current Protocols in Molecular Biology” (Wiley, New York, 1996); Perbal, “A Practical Guide to Molecular Cloning” (1984); the series, Methods In Enzymology (S. Colowick and N. Kaplan eds., Academic Press, Inc.); Handbook of Experimental Immunology, Vols. I-IV (D. M. Weir and C. C. Blackwell eds., 1986, Blackwell Scientific Publications).

Before the present nucleic and amino acid sequences, compositions, reagents, methods and uses thereof are described, it is to be understood that this invention is not limited to the particular sequences, compositions, reagents, methods and uses described herein as the skilled artisan will understand that these may vary and still be contained within the scope of the invention. It is also to be understood that the terminology used herein is for the purpose of describing particular embodiments only and is not intended to limit the scope of the present invention, which will be limited only by the claims appended hereto.

Except as noted in the description, all technical and scientific terms used herein have the same meaning as is commonly understood by one of ordinary skill in the art to which the invention applies. All publications mentioned herein are incorporated by reference herein for any purpose and in their entirety, including all text, figures, graphs, equations, illustrations, and drawings.

The present invention relates generally to methods for identifying virulence factors in microorganisms, particularly pathogenic bacteria that infect aquatic species, such as fish. As used herein, “virulence” refers generally to the ability of a microorganism to cause disease and the severity of the disease the microorganism causes, while “virulence factors” are generally those attributes of a microorganism that are responsible for the disease-causing ability of the microorganism. Virulence factors of the invention can include, for example, polynucleotide sequences of the microorganism and the polypeptides encoded thereby.

The present invention is based on the observation that virulence factors in marine pathogens, particularly bacterial pathogens of fish such as S. iniae, can be identified by transposon-mediated random mutagenesis. Thus, the present invention provides a method for identifying a virulence factor in a marine pathogen by randomly mutagenizing the marine pathogen and comparing the virulence of the resulting random mutants with the unmutagenized pathogen. Random mutants thus identified that are less virulent than the unmutagenized pathogen have mutations in a region necessary for the virulence of the pathogen. Identification of the mutagenized region, therefore, identifies the virulence factor.

As used herein, “random mutagenesis” or “randomly mutagenizing” refers to the process of generating mutations throughout a genome or along the length of a nucleic acid, generally without regard to position. However, certain mutagens may show a degree of preference for one type of nucleotide, sequence or location in the genome an organism (such as cytosine versus guanine, actively transcribed regions versus non-transcribed regions), without specifically targeting a particular sequence. Such mutation preferences are within the scope of “random mutagenesis” as contemplated by the present invention.

Mutagenesis according to the present invention can conveniently be accomplished using a transposon, such as Tn917, as described below in the Examples. Transposons have been known for more than three decades and have been used for insertion mutagenesis. See e.g., Tomich et al., J. Bacteriol. 141:1366-1374 (1980); Gutierrez et al., J. Bacteriol 178:4166-75 (1996). Thus, suitable transposons for use in the methods of the invention will be well known in the art.

The skilled artisan will also be aware of additional mutatgenesis methods that are well-known. Random mutagenesis according to the present invention may be performed by use of any suitable physical, chemical or biological mutagen, by use of suitable oligonucleotide (i.e. random primers), or by subjecting the DNA sequences of the pathogen to PCR generated mutagenesis. Furthermore, random mutagenesis may be performed by use of any combination of these mutagenizing agents.

Examples of physical and chemical mutagens suitable for use in the methods of the present purpose include but are not limited to ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues. Examples of biological mutagenesis methods include but are not limited to transduction and insertion by e.g., viruses, transposons and other insertion elements; recombination; and error-producing enzymatic mutagenesis.

The mutagenizing agent may, e.g., be one which induces transitions, transversions, inversions, scrambling, deletions, and/or insertions. In certain embodiments, the mutagenizing agent generates the insertion of a “signature” that can be used to identify the site of mutational insertion.

Mutations of the invention include insertions, deletions, substitutions and the like involving only a single nucleotide base or can involve multiple bases. Deletions of a single base, for example, can interrupt the reading frame of a coding region, thereby rendering the encoded protein non-functional or partially non-functional, while larger deletions can eliminate a part or all of the gene completely. The skilled artisan will recognize multiple mutation scenarios, each of which are encompassed by the invention, that can lead to deficiency in a bacterium.

Transposon mutagenesis is particularly useful because insertion of the transposable element not only disrupts and thereby inactivates genes, it also provides an anchor point for primed synthesis. As such, the transposable element can be used to locate and sequence the mutagenized gene. Thus, in one embodiment of the invention, a library of transposon insertion mutants of a marine pathogen, such as S. iniae is prepared. The transposon can be any known in the art that is effective in the pathogen of interest. For S. iniae, Tn917 can be used, as described in the Examples below.

The present invention also provides collections of mutagenize pathogens (libraries) that have been found to have reduced virulence in an aquatic species. A library of different avirulent pathogens may be useful as a heterogeneous live attenuated vaccine. In turn, individual avirulent bacteria can be isolated from the library and analyzed to determine the mutagenized virulence factor therein. In certain embodiments of the invention, the library contain pathogens mutagenized in at least one of a PGM, ABC transporter, integrase, recombinase, transposase, tRNA synthetase, or membrane protein gene. Isolated bacteria mutagenized in one of the aforementioned genes are also included in the invention.

Once virulence genes are identified as described above, site-directed mutations can be introduced into the wild type of the pathogen to generate additional mutations in the same gene, which may have different degrees of virulence as compared to the original identifying mutation. The skilled artisan will be aware of a variety of methods that are well known in the art for generating site-directed mutations, including deletions, insertions and substitutions of 1, 2, 3, 4, 5 or more nucleotides. In some embodiments, at least about 10 nucleotides are mutated. In other embodiments of the invention, at least about 50, at least about 100, at least about 250, at least about 500, at least about 1000 or more nucleotides in and/or around a virulence gene are mutated.

In certain aspects of the invention, directed mutations can be introduced by homologous recombination, for example by introducing vectors containing sequences from the virulence gene into the pathogen. In a typical embodiment, a plasmid is constructed to contain 5′ and 3′ sequences of a virulence gene flanking a selectable marker, in the same orientation as found in a bacterium. When introduced into the pathogen, the homologous regions of the plasmid and the bacterial chromosome undergo recombination and regions of the chromosomal virulence gene are exchanged for the selectable marker, thereby deleting the virulence gene and replacing it with the marker. Execution, adaptation and modification of this method of the invention are well within the skill in the art using only routine experimentation.

In one embodiment of the invention, the marine pathogen infects aquatic species, such as fish. In one aspect of the invention, the aquatic species is a human or animal food source, such as, for example, bass, trout, salmon, catfish, tilapia, plait, cod, halibut, carp, yellowtail, eel or sturgeon. In one aspect of the invention, the marine pathogen can infect tilapia (e.g., Oreochromis niloticus, Sarotherodon, spp.) and/or hybrid striped bass (e.g., Morone saxatilis×M. chrysops). In another aspect, the marine pathogen can infect channel catfish (e.g., Ictaluris punctatus), rainbow trout (e.g., Oncorhynchus mykiss and Oncorhynchus spp.), eel: (e.g., Anguilla spp.), yellowtail: (e.g., Seriola quinqueradiatia), turbot: (e.g., Scophthalinus maximus), and/or sea bass: (e.g., Dicentrarchus labrax). In certain embodiments, the aquatic species are farmed or raised by methods of aquaculture. In other embodiments of the invention, the aquatic species are pets or aquarium animals. It will also be understood that the same pathogens may at times also infect other species, such as human and other mammals. Thus, while the organisms of the invention may be pathogenic in fish, their host-range may not be limited to fish.

Typically, the marine pathogen is a bacterium. However, the methods of the present invention are adaptable to identification of virulence factors of a wide variety of marine pathogens including for example, viruses, fungi and parasites. In one embodiment of the invention, the marine pathogen is member of the Streptococcus genus, such as Streptococcus iniae. In other embodiments, the marine pathogen can be, for example, Streptococcus difficile, Lactococcus garvieae, Lactococcus piscium or Vagococcus salinoninarum.

The present invention also provides attenuated mutants of S. iniae. As used herein, “attenuated” refers to a reduction in the virulence of a pathogenic microorganism. Thus, an “attenuated mutant” is one which displays reduced virulence in at least one host for which it is pathogenic. Attenuated S. iniae mutants typically have reduced virulence in aquatic species, such as fish and particularly in hybrid striped bass (HSB) and/or tilapia. Attenuated mutants can be those identified through random transposon mutagenesis and direct screening for virulence in a aquatic species such a fish, for example a hybrid striped bass. In other embodiments of the invention, insertions, deletions and other disruptions can be introduced in a site-directed fashion once a virulence gene has been identified. In one aspect of the invention, the mutant is severely attenuated. For example, an entire gene can be deleted, replaced or rendered non-functional.

Certain mutants of the invention contain disruptions in an open reading frame (ORF) of the marine pathogen. As used herein, an “open reading frame” refers to a sequence of nucleotides that codes for a contiguous sequence of amino acids. ORFs of the invention may code for the amino acids of a polypeptide of interest from the N-termius of the polypeptide (typically a methionine encoded by a sequence that is transcribed as AUG) to the C-terminus of the polypeptide. ORFs of the invention include sequences that encode a contiguous sequence of amino acids with no intervening sequences (e.g., an ORF from a cDNA) as well as ORFs that comprise one or more intervening sequences (e.g., introns) that may be processed from an mRNA containing them (e.g., by splicing) when an mRNA containing the ORF is transcribed in a suitable host cell. ORFs of the invention also comprise splice variants of ORFs containing intervening sequences. The invention is contemplated to include not only the disruptions in virulence factors that are generated by random mutagenesis, but also directed mutations in the virulence factors that are identified by methods of the present invention. As such, the skilled artisan will recognize that a disruption of an open reading frame can be the result of an insertion, deletion, substitution or the like.

Furthermore, mutations of both a random and directed nature can be introduced into control sequences (e.g., promoters, repressor, operators and the like) of virulence factors, that reduce or eliminate the expression of the virulence factor without disturbing coding sequences.

In certain embodiments of the invention, mutations in a virulence factor can include the insertion of a selectable marker. As used herein, the phrase “selectable marker” refers to a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products that provide resistance against otherwise toxic compounds (e.g., antibiotics); (2) nucleic acid segments that encode products that are otherwise lacking in the recipient cell (e.g., tRNA genes, auxotrophic markers); (3) nucleic acid segments that encode products that suppress the activity of a gene product; (4) nucleic acid segments that encode products that can be readily identified (e.g., phenotypic markers such as (β-galactosidase, green fluorescent protein (GFP), yellow flourescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; and/or (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

In one embodiment of the invention, the ORF has similarity to bacterial phosphoglucomutase (pgm) genes. The enzyme phosphoglucomutase (PGM) interconverts glucose-6-phosphate and glucose-1-phosphate, and has recently been reported to play an important role in polysaccharide capsule production and virulence in a variety of Gram-positive and Gram-negative bacterial pathogens. Mutants of the invention in or affecting a pgm gene may produce a reduced amount or no phosphglucolmutase protein. In other aspects of the invention, mutants may produce, for example, variant, modified or truncated PGM proteins. Typically, the effect of pgm mutation is a deficiency in phosphglucolmutase activity.

In other aspects of the invention, the mutants contain an insertion in an ABC transporter, an integrase, a recombinase, a tRNA synthetase, a transposase, or a membrane protein of the marine pathogen.

In other embodiments of the invention, the mutations are the result of deletion and/or insertion by recombination, including but not limited to, homologous recombination as described in Example 7 below.

The attenuated mutants of the invention can be associated with additional phenotypic changes in the marine pathogen. In one embodiment of the invention, mutations in pgm of S. iniae are associated with alterations in cell wall morphology, cell hydrophobicity, cell volume, surface charge, biding to cytochrome C, cell buoyancy, capsule production, and susceptibility to innate immune defenses.

In certain embodiments of the invention, attenuated mutants display reduced virulence, for example in an aquatic species. According to one aspect of the invention, mutation of pgm can be associated with markedly reduced virulence of S. iniae. For example, mortality associated with S. iniae may be reduced by at least about 90% in attenuated mutant forms. In other aspects of the invention, mortality may be reduced to zero.

In yet another embodiment, attenuated mutants may induce protective immunity in aquatic species against wild-type S. iniae. As such, prior infection with an attenuated mutant may reduce or eliminate subsequent pathogenic infection by wild-type S. iniae. In some case, infection with 10 fold, 100 fold, 1000 fold, 10000 fold or more wild-type S. iniae may be required before a pathogenic effect is observed in an animal previously immunized with by exposure to an attenuated mutant.

Also provided by the invention is an isolated Streptococcus iniae bacterium containing a phosphglucolmutase deficiency. In certain aspects, phosphglucolmutase enzyme activity is decreased at least 10-fold in the S. iniae of the invention compared to a wild type Streptococcus iniae bacterium. As illustrated in the Examples below, deficiency in phosphglucolmutase can result from a mutation in a gene encoding a phosphglucolmutase. The mutation can be any mutation in a gene encoding a phosphglucolmutase protein that results in phosphglucolmutase deficiency of the organism. For example, the mutation can be an insertion that interrupts a phosphglucolmutase ORF or it can be a deletion of all or part of a phosphglucolmutase gene. In certain embodiments, the mutation is an insertion, such as an insertion of a transposon. In one aspect of this embodiment, the transposon is Tn917.

The invention further contemplates that a mutation in a sequence that regulates the expression of a phosphglucolmutase gene can result in phosphglucolmutase deficiency and therefore such mutants are encompassed by the present invention. Sequences that regulate the expression of a phosphglucolmutase gene can include, but are not limited to, promoters, repressors, operators and the like.

The isolated phosphoglucomutase-deficient bacterium will typically comprise at least one phenotypic difference when compared to a wild type bacteria. The phenotypic differences can be decreased buoyancy; increased cell hydrophobicity; decreased surface associated exopolysaccharide capsule; decreased binding to cytochrome C; decreased surface negative charge; and increased cell volume. Certain phosphoglucomutase-deficient bacteria of the invention display all of these phenotypic differences. In one embodiment, the phosphoglucomutase-deficient bacterium of the invention is a S. iniae that is phenotypically distinguishable from wild-type S. iniae.

An isolated phosphoglucomutase-deficient bacterium of the invention may produce a reduced amount of phosphoglucomutase (PGM) protein or may produce no PGM at all. Other phosphoglucomutase-deficient bacteria of the invention produce altered, variant, mutant or truncated phosphoglucomutase proteins. In some cases, the mutation is incapable or inefficient at converting glucose-1-phosphate to glucose-6-phosphate. Typically, the phosphoglucomutase mutations of the invention are avirulent, particularly in fish, such as HSB.

Also provided by the present invention are polynucleotide and polypeptide sequences of S. iniae phosphoglucomutase. The term “polynucleotide” is used broadly herein to mean a sequence of two or more deoxyribonucleotides or ribonucleotides that are linked together by a phosphodiester bond. As such, the term “polynucleotide” includes RNA and DNA, which can be a synthetic RNA or DNA sequence, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide useful as a test agent can contain nucleoside or nucleotide analogs, or a backbone bond other than a phosphodiester bond.

In general, the nucleotides comprising a polynucleotide are naturally occurring deoxyribonucleotides, such as adenine, cytosine, guanine or thymine linked to 2′ deoxyribose, or ribonucleotides such as adenine, cytosine, guanine or uracil linked to ribose. However, a polynucleotide also can contain nucleotide analogs, including non naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker & Crooke, BioTechnology 13:351360, 1995, each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

The term “polypeptide” is used broadly herein to mean two or more amino acids linked by a peptide bond. Generally, a polypeptides useful in methods of the invention contain at least about two, three, four, five, or six amino acids, and can contain about ten, fifteen, twenty or more amino acids. As such, it should be recognized that the term “polypeptide” and “peptide” are used interchangeably and are not used herein to suggest particular sizes or numbers of amino acids, and that a polypeptide or peptides of the invention can contain several amino acid residues or more. Polypeptides and peptides of the invention can be prepared, for example, by a method of chemical synthesis, or can be expressed from a polynucleotide using recombinant DNA methodology. Where chemically synthesized, polypeptide and peptides containing one or more D-amino acids, or one or more amino acid analogs, for example, an amino acid that has been derivatized or otherwise modified at its reactive side chain, or in which one or more bonds linking the amino acids or amino acid analogs is modified, can be prepared. In addition, a reactive group at the amino terminus or the carboxy terminus or both can be modified. Such peptides can be modified, for example, to have improved stability to a protease, an oxidizing agent or other reactive material the peptide may encounter in a biological environment, and, therefore, can be particularly useful in performing a method of the invention. Of course, the peptides can be modified to have decreased stability in a biological environment such that the period of time the peptide is active in the environment is reduced.

As described below, a transposon-insertion mutant of S. iniae resulting in phosphoglucomutase-deficiency (TnM2) was found to be attenuated and avirulent. Sequence analysis of the phosphoglucomutase affected in TnM2 revealed the pgmS1 gene, as set forth in SEQ ID NO:5. The polynucleotide sequence is predicted to encode a PGM polypeptide (SEQ ID NO:6) with significant homology to known bacterial PGMs, particularly Streptococcal PGMs.

The present invention demonstrates that PGM is a virulence factor of S. iniae. Polypeptides of the invention, specifically polypeptides having the amino acid sequence set forth in SEQ ID NO:6 and immunogenic fragments thereof, may be useful for raising diagnostic and therapeutic antibodies, generating an immune response to S. iniae, and for the production of subunit vaccines against S. iniae.

However, live vaccines are generally thought to generate immune responses of greater magnitude and of longer duration than those produced by killed or subunit vaccines. A single dose of a live-attenuated vaccine can provide better protection against later infection by the wild-type organism, because the attenuated organism persists and metabolizes within the host, and in some cases may replicate in the host for a time.

As used herein, “vaccine” is defined in a broad sense to refer to any type of biological agent in an administratable form capable of stimulating a protective immune response in an animal. In certain embodiments, a vaccine of the invention can comprise live or killed cells, fractions or extracts of cells, isolated molecules derived from cells, such as polypeptides or immunogenic fragments of polypeptides, and combinations thereof. “Subunit vaccine” as used herein refers to a vaccine comprising individual molecules or fragments thereof. Vaccines and particularly subunit vaccines can be comprised of recombinant or synthetic polynucleotides molecules, recombinant or synthetic polypeptides and the like.

The present invention provides such an attenuated live vaccine comprising an attenuated S. iniae mutation, such at the ΔPGM mutant, TnM2. TnM2 displays the ability to generate an immune response that is protective against wild type S. iniae infection, thereby preventing meningoencephalitis disease and death in up to 100% of vaccinated hybrid striped bass.

Live vaccines offer the additional advantage of adaptability. While most subunit and killed vaccines must be injected, live vaccines can be administered by a variety of routes. For example, live vaccines can be administered by oral feeding (e.g., in food) or immersion of aquatic species, in addition to injection (subcutaneous, intravenous, intramuscular, intraperitoneal, etc.). Thus, the present invention provides a vaccine for S. iniae disease prevention comprising an isolated S. iniae bacterium, particularly a live S. iniae bacterium, with a phosphoglucomutase deficiency.

The skilled artisan will appreciate that the dosage of live attenuated S. iniae vaccine required to protect aquatic species against S. iniae disease will depend on such variables as the route of administration and size of the animal vaccinated, and can be determined using routine experimentation. Similarly, the skilled artisan will recognize that it may be advantageous to prepare pharmaceutical or veterinary compositions comprising the live-attenuated vaccines and additional ingredients for example, to stabilize, preserve, disperse or facilitate administration of the live vaccine. Thus, it is contemplated that vaccine compositions of the present invention may include suitable carriers, adjuvants, stabilizers and/or diluents.

The present invention also provides methods for preventing Streptococcus iniae disease in a subject, such as a aquatic species, such as a fish (e.g., HSB or tilapia) by administering an isolated S. iniae bacterium comprising a phosphoglucomutase deficiency, such as the transposon insertion mutants described herein. According to this method of the invention, exposure to the attenuated bacterium evokes immunity to the wild type bacterium, thereby preventing or minimizing S. iniae disease.

It will be appreciated that the methods and compositions of the invention would be equally applicable using S. iniae attenuated with respect to other virulence factors, such as those identified in Table 1, below, which are also contemplated by the present invention.

The following examples are provides solely for the purpose of illustrating embodiments of the invention and are not intended to impose limitations thereupon.

### EXAMPLE 1

### Materials & Methods

### Bacteria Strains, Culture, Transformation, and DNA Techniques

Wild-type (WT) S. iniae strain K288 was isolated from the brain of a diseased HSB using standard microbiological techniques. K288 was identified unambiguously as S. iniae through biochemical testing and by analysis of ribosomal 16 s sequences, and proven to be virulent in an HSB model by intraperitoneal (IP) injection of 4×10⁵ cfu of the bacterium, a lethal dose in all fish tested. S. iniae were propagated in Todd-Hewitt broth (THB) or on Todd-Hewitt agar (THA) at 30° C. unless otherwise indicated, with antibiotic selection of 2 μg/ml chloramphenicol (Cm), 5 μg/ml erythromycin (Em) and 500 μg/ml kanamycin (Kan) when required. E. coli were grown in Luria-Bertani (LB) medium at 37° C. using 500 μg/ml of Em, 15 μg/ml of Cm, or 100 μg/ml of ampicillin (Amp) for selection unless otherwise indicated. S. iniae were rendered competent for electroporetic transformation by growth in THB plus 0.6% glycine following procedures reported for group B Streptococcus. See Framson et al., App. Environ. Microbiol. 63:3539-3547 (1997). Transformants were identified by recovery for 2 h at 30° C. in SOC medium for E. coli and THB plus 0.25 M sucrose for S. iniae, followed by plating on appropriate antibiotic selective media. For use in fish challenges, overnight cultures of S. iniae were diluted 1:20 and grown to mid-log phase (OD 600 nm=0.4) corresponding to approximately 4×10⁸ colony-forming units (cfu)/ml. Plasmid DNA was isolated from E. coli using QiaPrep kits (Qiagen, Valencia, Calif.). For S. iniae, a 15 min. incubation at 37° C. in 100 U of mutanolysin (Sigma, St. Louis, Mo.) preceded plasmid isolation using the QiaPrep kit. Genomic DNA was isolated using the UltraClean™ DNA Isolation Kit (MoBio, Carlsbad, Calif.).

### EXAMPLE 2

### Identification of Attenuated Mutants OF S. iniae

### Transposon Mutagenesis.

Transposon mutagenesis of strain K288 followed procedures described for S. mutans using the temperature-sensitive plasmid pTV₁OK bearing transposon Tn917, (Gutierrez et al., J. Bacteriol. 178:4166-75 (1996)) with slight modifications. Individual colonies of K288 transformed with pTV₁OK were inoculated into THB+Kan and grown to OD₆₀₀=0.9 at the permissive temperature (30° C.) for plasmid replication. Cultures were diluted 1:100 in THB+Em and grown at a nonpermissive temperature (37° C.) to an OD600=0.9, then plated on THA+Em for isolation of candidate insertion mutants. Libraries of S. iniae mutants bearing random transposon insertions into the genome were verified by Southern blot analysis.

### In Vivo Screening of Transposon Mutants for Loss of Virulence

HSB (Morone chryosops×Morone saxitilis) aged <1 year and with an average weight of approximately 30 g were used for an in vivo challenge model of S. iniae infection. HSB were challenged intraperitoneally (IP) with 100 μl of WT or mutant S. iniae bacteria resuspended in PBS at known inocula and injected using a 27 ga. needle. Fish were held with aeration and flow-through water at 24-27° C. for 7 days after challenge, and were monitored twice daily for mortalities. Brain biopsy cultures were taken from selected mortalities to confirm infection with the challenge S. iniae strain based on the appropriate antibiotic sensitivity profile. Screening the transposon library in this manner revealed numerous attenuated mutants listed below in Table 1, including mutant TnM2 with a transposon disruption of a putative pgm gene.

TABLE 1
Exemplary Attenuated Mutants Identified by Transposon Disruption

| | Putative disrupted gene |
|---|---|
| Mutant name | (based on similarity to known gene sequences) |
| TnM1 | ABC transporter (SEQ ID NO: 11) |
| TnM2 | Phosphoglucomutase (SEQ ID NO. 5) |
| TnM3 | Unknown |
| TnM4 | Unknown |
| TnM5 | Phosphoglucomutase |
| TnM6 | Unknown |
| TnM7 | Unknown |
| TnM8 | Phosphoglucomutase |
| TnM9 | Unknown) |
| TnM10 | Unknown |
| TnM11 | Unknown |
| TnM12 | Unknown |
| TnM13 | Unknown |
| TnM14 | Unknown |
| TnM15 | Unknown |
| TnM16 | Unknown |
| TnM17 | Unknown |
| TnM18 | Unknown |
| TnM19 | Phosphoglucomutase |
| TnM20 | Integrase/recombinase |
| TnM21 | Unknown |
| TnM22 | Unknown |
| TnM23 | ABC transporter |
| TnM24 | Unknown |
| TnM25 | Unknown |
| TnM26 | Unknown |
| TnM27 | Unknown |
| TnM28 | Unknown |
| TnM29 | Unknown |
| TnM30 | tRNA synthetase |
| TnM31 | Unknown |
| TnM32 | Unknown |
| TnM33 | Unknown |
| TnM34 | Unknown |
| TnM35 | Unknown |
| TnM36 | transposase in exopolysaccharide gene cluster |
| TnM37 | Unknown |
| TnM38 | Unknown |
| TnM39 | Unknown |
| TnM40 | Unknown |
| TnM41 | Unknown |
| TnM42 | Unknown |
| TnM43 | Unknown |
| TnM44 | Unknown |
| TnM45 | Hypothetical membrane protein |
| TnM46 | Unknown |
| TnM47 | Unknown |
| TnM48 | Unknown |
| TnM49 | Unknown |

The Tn917 insertion site in the attenuated mutant TnM2 was identified by direct sequencing from genomic DNA using the modified primer (fimer) 5′-GAAACATTGGTTTAGTGGGAATTTGTAC-3′ (SEQ ID NO:3) and 0.1 μl of ThermoFidelase (Fidelity Systems, Gaithersburg, Md.) in a 20 μl cycle sequencing reaction (Big Dye® v3.0; Applied Biosystems Inc). Chromosome walking using a single-primer PCR technique (Hermann et al., Biotechniques 29:1176-8, 1180 (2000); Karlyshev et al., Biotechniques 28:1078, 1080, 1082 (2000)) was used to determine the entire sequence of pgm and flanking regions. Sequence files were analyzed with Chromas software (Technelysium, Tewantin, Australia), aligned with BioEdit sequence alignment editor (Ibis Therapeutics, Carlsbad, Calif.), and annotated with Artemis v5.0 DNA sequence viewer (Sanger Institute, Cambridge, UK). The amino acid sequence of PGM was compared to sequences in the Genbank databases using the BlastP program (Altschul et al., J. Mol. Biol. 215:403-410 (1990)).

### Results

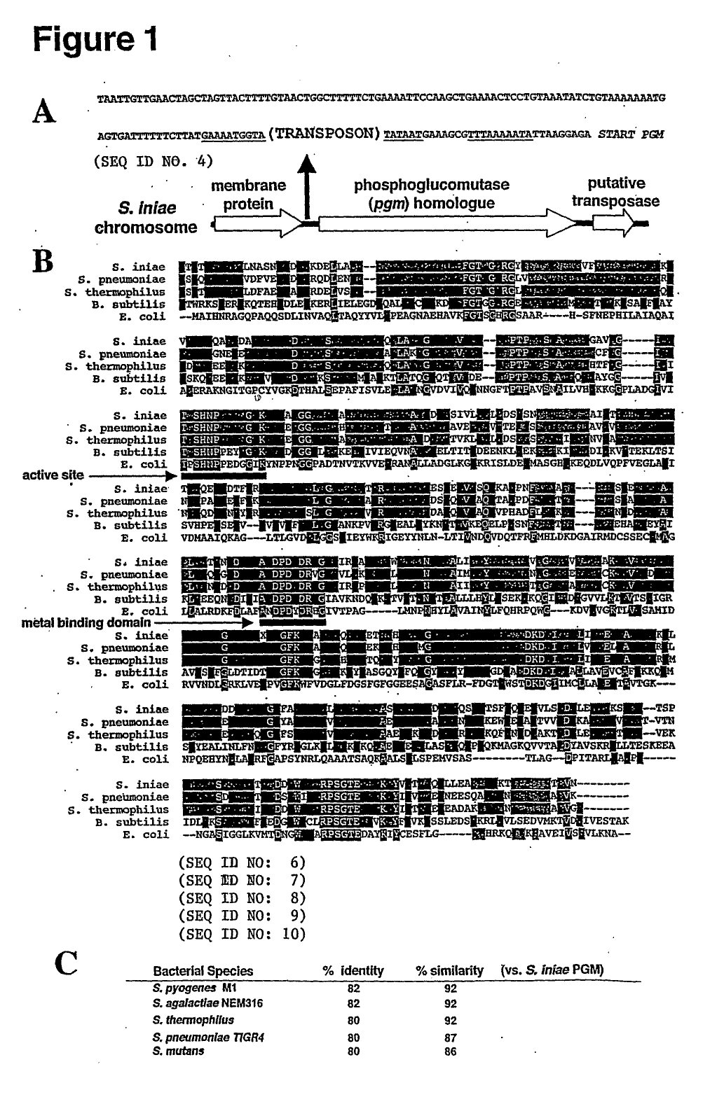

The attenuated mutant TnM2 was identified in a screen of a Tn917 chromosomal insertion library of WT S. iniae strain K288 for loss of virulence in HSB. Using direct sequencing and single-primer chromosome walking, the site of the sole Tn917 insertion in TnM2 and surrounding DNA sequence was determined (FIG. 1A; SEQ ID NO:4). Tn917 was found to have inserted in a predicted promoter region 32 bp upstream of the start ATG codon of the ORF sharing strong sequence homology to genes encoding PGM enzymes. Herein this ORF will be designated pginSI (Genbank nucleotide sequence accession number AY846302; SEQ ID NO:5; BlastP analysis in GenBank of the deduced amino acid sequence of the candidate S. iniae PGM (Genbank protein sequence accession number AAW56093; SEQ ID NO:6) revealed strong sequence homology and identity with known α-PGM enzymes from other Gram-positive bacterial species (FIG. 1B; SEQ ID NOs:7-10), as well as several known α-PGMs from Gram-negative and vertebrate species. In particular, the functional regions characteristic of α-PGM proteins were conserved in the S. iniae homologue including a metal binding domain and active site residues. See Dai et al., J. Biol. Chem. 267:6322-6337 (1992).

### EXAMPLE 3

### Enzymatic Activity OF TnM2

### Phosphoglucomutase Activity Assays.

Cells extracts were prepared from bacteria in mid-log phase (OD₆₀₀ nm=0.4), collected by centrifugation and washed twice in chilled phosphate buffered saline (PBS). For E. coli, cells were resuspended in 50 mM TEA (triethanolamine) buffer with 5 mM MgCl₂ (pH 7.2) and suspensions frozen at −80° C. Cell lysates were prepared using sonication (3 bursts of 10 seconds each). For S. iniae, cell pellets were frozen. Cells lystaes were prepared using the CelLytic™ B Plus cell lysis reagent (Sigma Aldrich, St Louis, Mo.) following the manufacturers instructions. All cell lysates were centrifuged (30 minutes at 10000×g) and the supernatants (crude cell extracts) were stored at −80° C. until use. The specific activity of PGM in cell extracts was measured as the conversion of α-glucose-1-phosphate to glucose-6-phosphate in a reaction coupled to reduction of glucose-6-phosphate by glucose-6-phosphate dehydrogenase, and quantitated spectrophotometrically at 340 nm by monitoring the formation of NADPH from NADP⁺ at 30° C. The PGM assay solution contained final concentrations of 5 mM MgCl₂, 0.4 mM NADP⁺, 2 U glucose 6-phosphate dehydrogenase, and 50 μM α-glucose 1,6-bisphosphate. After the cell extract was added, the reaction was initiated with addition of α-glucose-1-phosphate to 1.4 mM. All chemicals for the phosphoglucomutase activity assays were obtained from Sigma Aldrich (St. Louis, Mo.).

Complementation and Heterologous Expression of S. iniae PGM.

The entire PGM gene plus the upstream putative promoter region (1865 bp) was amplified from WT S. iniae K288 genomic DNA using forward primer 5′-GAACTAGCTAGTTACTTTTGTAACTG-3′ (SEQ ID NO:1) and reverse primer 5′-CTAATTCACAAAAGTGTTGATTTCAG-3′ (SEQ ID NO:2) in a standard PCR reaction using Platinum® PCR SuperMix (22 U/ml complexed recombinant Taq DNA polymerase with Platinum® Taq Antibody, 22 mM Tris-HCl (pH 8.4), 55 mM KCl, 1.65 mM MgCl2, 220 μM dGTP, 220 μM DATP, 220 μM dTTP, 220 μM dCTP; Invitrogen, Carlsbad, Calif.) and 30 cycles of denaturation (94° C., 30 sec.), annealing (55° C., 30 sec.), and elongation (72° C., 1.5 min.). The resulting product was T-A cloned into the pCR® 2.1-TOPO®. vector (Invitrogen, Carlsbad, Calif.). The pgm gene was cut from the pCR® 2.1-TOPO® construct with BamH1 and Xba1 and cloned into the corresponding sites in the E. coli-streptococcal shuttle expression vector pDC123 (Chaffin & Rubens, Gene 219:91-9 (1998)) bearing Cm^{R} to create pSiPGM. This recombinant vector was used to transform WT S. iniae K288, S. iniae transposon mutant TnM2, and an E. coli K-12 ΔPGM mutant (Adhya & Schwartz, J. Bacteriol. 108:621-6 (1971)); similar transformations were performed with pDC123 to serve as a vector only control. Transformants were identified by Cm^{R} and confirmed by restriction enzyme digestion and PCR analysis of plasmid preparations from transformed cells.

Results: PGM Activity in S. iniae is Linked to pgmSI

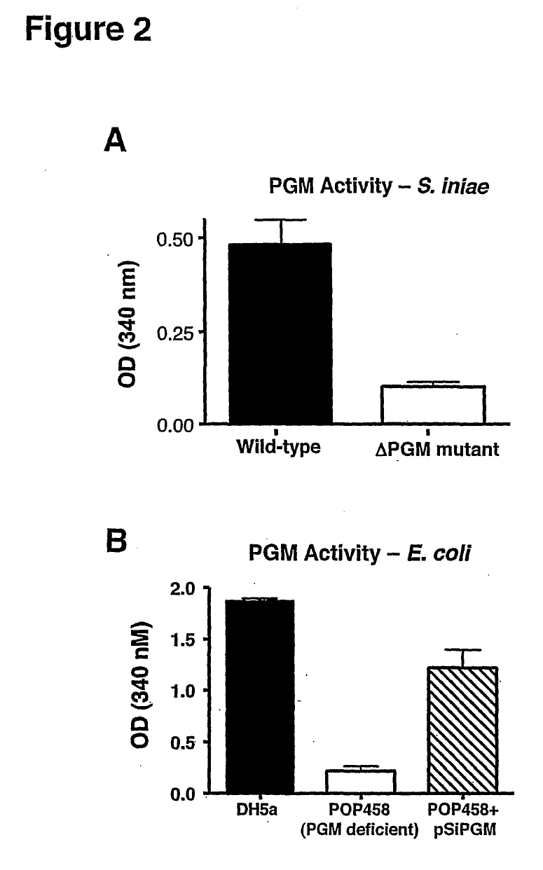

Biochemical assays were performed to confirm that the pgmSI ORF encoded a functional PGM enzyme. Measured PGM activity was decreased 10-fold in mutant TnM2 compared to the WT S. iniae parent strain (FIG. 2A). Complementation of TnM2 with plasmid pSiPGM restored PGM activity to approximately WT levels. A mutant strain of E. coli (Pop458) with a disrupted native PGM gene has greatly reduced PGM activity compared to WT is. coli. Heterologous expression on pSiPGM in E. coli Pop458 significantly increased PGM activity (P<0.0001) (FIG. 2B). Together these studies demonstrate that pgmSI is both necessary and sufficient for PGM enzymatic activity.

### EXAMPLE 4

### Virulence of S. iniae Mutants

### Fish Virulence Studies.

Groups of 40 HSB (approximately 30 g) were challenged intraperitoneally (IP) with 2×10⁵-2×10⁸ cfu of WT or mutant S. iniae bacteria from log phase (OD₆₀₀=0.4) cultures resuspended in 100 μl of PBS. Fish were held with aeration and flow-through water at 24-27° C. for 14 days after challenge, and were monitored twice daily for mortalities. To compare the virulence of the complemented mutant TnM2[pSiPGM] to the WT and TnM2 strains, groups of 28 fish for each treatment were injected with 4×10⁵ cfu and monitored for 7 days. Three fish were selected at random from each group at 24 hours post-challenge, sacrificed, and the cfu of S. iniae/ml of blood enumerated by plating dilutions on THA.

Results: S. iniae PGM Expression is Linked to Virulence.

The HSB challenge model was used to evaluate the virulence potential of S. iniae mutant TnM2 with reduced PGM activity. Whereas 100% of HSB injected with 4×10⁵ WT S. iniae died of meningoencephalitis, mortality was absent in fish challenged with TnM2 at the same dose and only 2.5% in fish challenged with the mutant at 1,000-fold higher inoculum (P<0.0001) (Table 2).

TABLE 2
Attenuation of Streptococcus iniae ΔPGM mutant (TnM2) in a hybrid
striped bass infection model

| Bacterium | Dose (cfu) | No. of fish | % Mortality |
|---|---|---|---|
| WT S. iniae | 4 × 10⁵ | 40 | 100 |
| PGM mutant | 4 × 10⁵ | 40 | 0 |
| | 4 × 10⁶ | 40 | 0 |
| | 4 × 10⁷ | 40 | 0 |
| | 4 × 10⁸ | 40 | 2.5 |

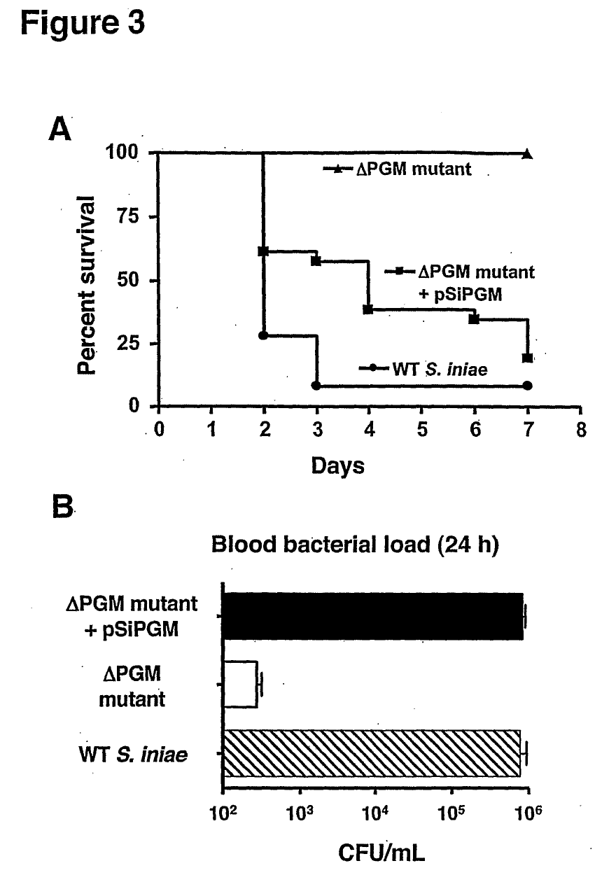

In a separate experiment, survival curves were plotted for HSB after intraperitoneal challenge with either 4×10⁵ cfu of WT S. iniae, mutant TnM2, or TnM2 complemented with pSiPGM. While no mortality was seen in fish infected with TnM2, reintroduction of the pginSl gene to the mutant on a plasmid vector resulted in mortality comparable that observed with WT S. iniae (FIG. 3A). Measurement of bacterial load in the blood of infected fish at 24 hours appeared to predict mortality. Bacterial levels in the blood of HSB infected with WT S. iniae or TnM2[pSiPGM] were 1,000 fold higher than those observed in TnM2 (FIG. 3B). The severe attenuation of mutant TnM2 in animal challenges identifies the pgm SI gene to be associated with one or more virulence phenotypes of S. iniae.

### EXAMPLE 4

### Phenotypic Characterization of S. iniae Mutants

### Assays for Buoyancy, Surface Charge, and Hydrophobicity.

To measure buoyancy, sequential overlay gradients of 1 ml each of 70%, 60% and 50% Percoll were prepared in 5 ml glass test tubes. One ml of overnight bacterial culture was placed on top of the Percoll layers, the tubes were centrifuged in a swinging bucket centrifuge for 8 min. at 500×g, and the migration of the bacteria to various Percoll interphases recorded. To measure surface charge, bacteria were grown to stationary phase, harvested by centrifugation, and washed twice in morpholinepropanesulfonic acid (MOPS) buffer (20 mM, pH 7.0). Cells were resuspended in MOPS buffer to an optical density at 600 nm (OD₆₀₀) of 6.0, and cytochrome C (Sigma, St. Louis, Mo.) was added to a final concentration of 0.5 mg/mL. After 15 min incubation at 23° C., samples were centrifuged (13,000×g for 5 min) and the amount of cytochrome C remaining in the supernatant was quantitated spectophotometrically at 530 nm. To measure hydrophobicity, stationary phase cultures were washed twice in PBS and resuspended in PBS at OD₆₀₀=10. 300 μl of n-hexadecane was layered on top of the cell suspension and the tubes were vortexed for 60 sec. Samples were incubated for 30 min at 23° C. to allow for phase separation. The aqueous phase was removed and the OD₆₀₀ was recorded to determine the quantity of bacteria remaining.

### Electron Microscopy.

S. iniae were grown to late-log phase (OD=0.7 at 600 nm) and collected by centrifugation. Pellets were resuspended in 0.07 M cacodylate buffer and transferred to 2.0 ml microfuge tubes. Samples were washed twice (0.007 M cacodylate buffer), resuspended in 0.2 M cacodylate plus 3.6% glutaraldehyde, incubated on ice for 1 hour, then washed three additional times. Polycationic ferritin (Sigma) was added to a final concentration of 1.0 mg/ml, the samples incubated for 30 min, washed twice, then stored at 4° C. Prior to electron microscopy, bacteria were washed three times in 0.1M sodium phosphate buffer (pH 7.3), post-fixed for 1 hour in 0.1 M phosphate buffered 2% osmium tetroxide, and rinsed three times in dH₂O. Dehydration was performed using ethanol at 30%, 50%, 70%, 95%, and 100% concentrations. Bacteria were immersed in two rinses of propylene oxide, and incubated for 2 hours in a mixture of 50% propylene oxide and 50% epoxy resin. Mollenhaure's formulation of Epon-Araldite was used to embed bacteria for thin sectioning. Sixty nanometer sections were placed on 300-mesh copper support grids and viewed using a Zeiss EM 10 electron microscope at an acceleration voltage of 80 kV using 16K and 50K magnifications. Negatives were enlarged and printed to final magnifications of 35K and 109K.

Results: S. iniae PGM Mutants have an Altered Cell Phenotype.

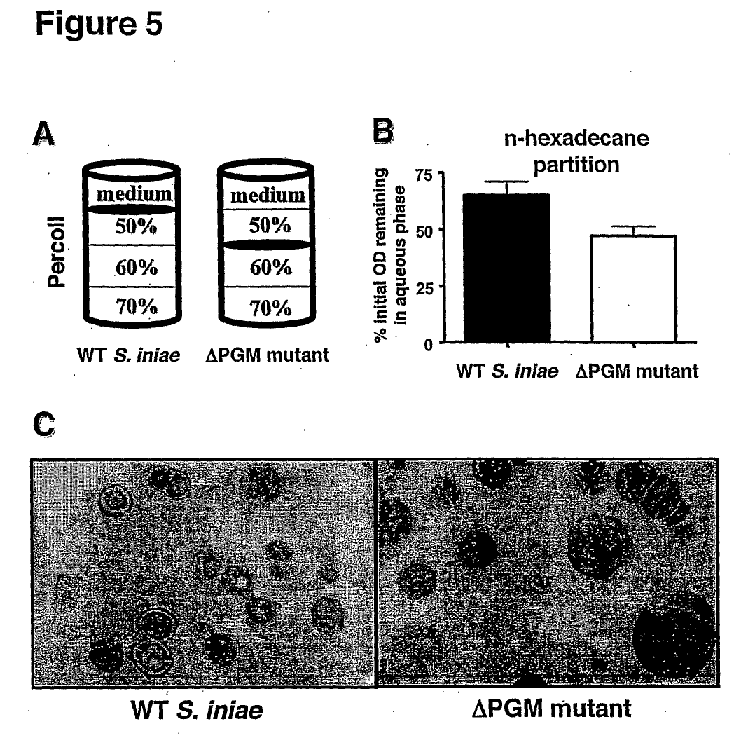

Assays were employed to identify general cell characteristics associated with loss of PGM activity in S. iniae. Logarithmic phase growth of WT S. iniae and the ΔPGM mutant in THB were equivalent. Migration through a Percoll gradient was increased in the ΔPGM mutant TnM2 compared to the WT S. iniae strain, indicative of a decrease in buoyancy (FIG. 5A). More ΔPGM mutant cells were found to partition from the aqueous phase into n-hexadecane than did WT S. iniae cells, consistent with an overall increase in cell hydrophobicity (FIG. 5B). The ΔPGM mutant also bound less cytochrome C than did the WT S. iniae strain, indicating that the mutant possessed a net decrease in surface negative charge. Our transmission electron microscopic analysis of WT and ΔPGM mutant S. iniae labelled with polycationic ferritin revealed that the mutant exhibited decreased amounts of surface-associated exopolysaccharide capsule (Barnes et al., Aquat. Organ. 53:241-7 (2003)), and that the average cell volume of ΔPGM mutant bacteria was 3 to 5 times larger than that of the WT strain (FIG. 5C).

### EXAMPLE 5

### Immune Response to ΔPGM Mutant S. iniae

### Blood Survival Assay.

Mid-log phase S. iniae suspensions of approximately 100 cfu in 100 μl PBS were added to 300 μl of fresh heparinized HSB blood in 2 ml siliconized plastic tubes and incubated for 1 hour at 30° C. on an orbital shaker. After 1 hour shaking, 100 μl aliquots were taken from each sample in duplicate and plated on THA for enumeration of surviving bacteria. Survival ratio was calculated as the cfu recovered from the sample divided by the initial inoculum. Each experiment was repeated three times, each time using the blood of a different HSB.

### Antimicrobial Peptide Sensitivity.

Early log phase cultures of S. iniae (OD₆₀₀=0.2) were diluted in fresh THB to approximately 2×10⁵ cfU/ml. 180 μl of this bacterial suspension was added to replicate wells of a 96-well plate. Dilutions of the antimicrobial peptides moronecidin (2 μM) (from HSB) and CRAMP (16 μM) (from mouse) were prepared in dH₂O and added to wells in 20 μl of distilled H₂O; dH₂O alone was used as a negative control. To measure antimicrobial killing kinetics, 20 μl aliquots from each well were serially diluted in PBS and plated at specified time points after addition of the antimicrobial peptide for cfa determination. Each experiment was performed in triplicate.

Characterization of the S. iniae Infection Process.

Hybrid striped bass (HSB; Morone chryosops×Morone saxitilis) aged <1 year and with an average weight of approximately 20 g were used to characterize the attenuation infection process of mutant TnM2. Groups of 50 HSB were challenged by IP injection of 2.5×10⁵ cfu/100 μl PBS of log-phase WT or TnM2 S. iniae. Fifty control fish were injected with 100 μl of PBS. Fish were held with aeration and flow-through water at 24-27° C. after challenge. Three fish were sacrificed at various time points during the first 124 hours of infection and bacterial load in the brain, blood and spleen was calculated by weighing tissue samples and homogenizing tissue samples in PBS (Tissue Tearor™, Biospec Products, Bartlesville, Okla.). Serial dilutions of each tissue homogenate in PBS were plated in duplicate on blood agar for enumeration of cfu. All TnM2 bacteria recovered were confirmed to have maintained the EmR phenotype. Brain and spleen tissues from each of the three fish sampled at 96 hours were pooled and placed in 10% buffered formalin until processing. Tissue pools were trimmed into cassettes, embedded in paraffin, and sections cut for routine histology. Serial sections were stained with either hematoxylin and eosin (H&E) or Giemsa, all sections were examined in a blinded fashion and scored as the number of positive traits seen in the sections of the tissue examined. H&E stained sections were used to score the respective spleen and brain changes while Giemsa stained sections were used to subjectively quantify the degree of bacteria associated with splenic ellipsoids and the meninges, respectively. The following criteria were used to assess the splenic response to infection: congestion, capsular hypertrophy, peritoneal inflammation, and ellipsoidal degeneration. Criteria for brain changes include meningeal inflammation and ventricle (optic lobe) inflammation.

Results: Sensitivity of the S. iniae ΔPGM Mutant to Innate Immune Clearance Mechanisms

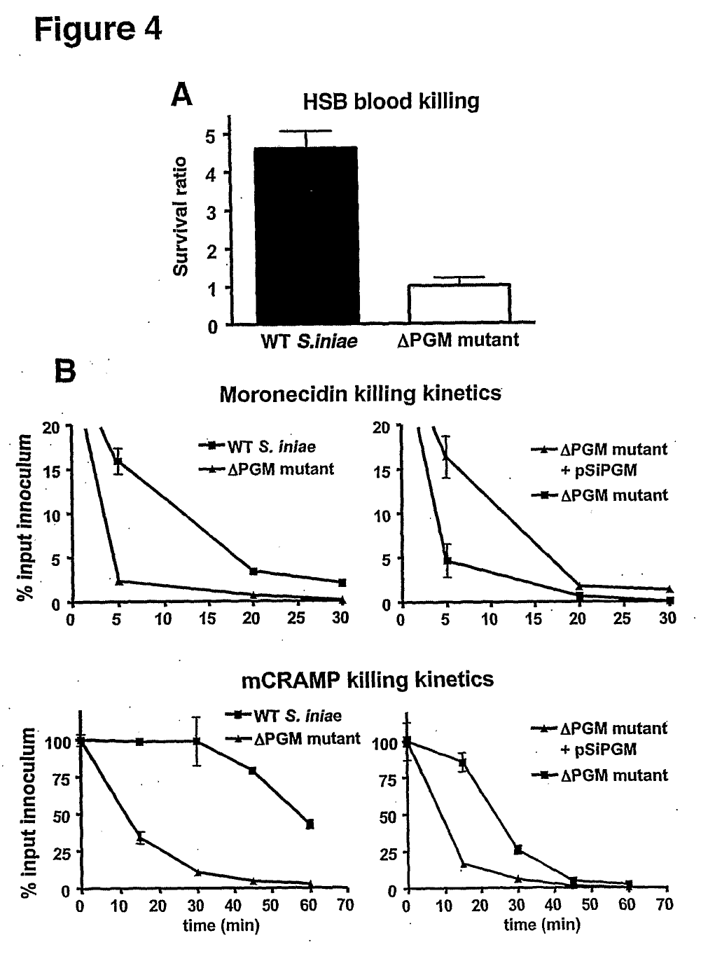

To measure the relative susceptibility of WT S. iniae and the ΔPGM mutant TnM2 to phagocytic clearance, a survival assay was performed in fresh blood isolated from HSB. Whereas the WT strain proliferated markedly in HSB blood, mutant TnM2 failed to do so (FIG. 4A). Cationic antimicrobial peptides (AMPs) have been recognized to be important components of innate defense and phagocyte killing in higher organisms. See Zasloff, Nature 415:389-95 (2002). HSB produce the AMP moronecidin (aka piscidin) possessing broad-spectrum antimicrobial activity. See Lauth et al., J Biol Chem 277:5030-9 (2002); Silphaduang & Noga, Nature 414:268-9 (2001). The ΔPGM mutant TnM2 was found to be significantly more sensitive than the WT S. iniae strain to moronecidin; similar results were seen with the murine cationic AMP mCRAMP. In vitro killing kinetics for each AMP showed that the rate of killing of the ΔPGM mutant was accelerated significantly compared to the WT S. iniae strain, whereas complementation of the mutant with pSiPGM produced a commensurate delay in the time course of AMP killing (FIG. 4B).

Analysis of the Aborted Infectious Process of ΔPGM Mutant S. iniae.

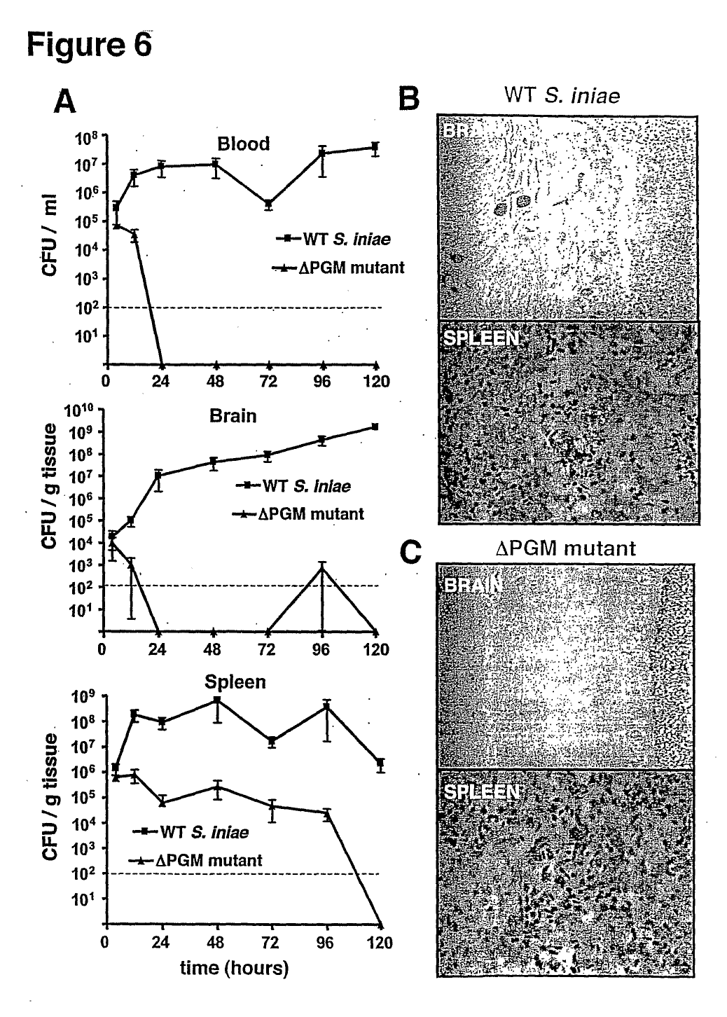

To further characterize the basis for the severely attenuated virulence of PGM-deficient S. iniae, we followed the course of infection in HSB challenged with WT and ΔPGM mutant S. iniae. At 4 hours post-infection, WT bacteria were found at 10⁵ cfu/ml in blood, >10⁴ cfu/g in brain, and approximately 10⁶ cfu/g in spleen (FIG. 6A). Bacterial counts in WT S. iniae-infected fish increased steadily over time, surpassing 10⁷ cfu/g in all tissues between 24 and 96 h of infection; widespread mortalities were observed. Histological examination of brain (48 h, H&E stain, 20×) revealed severe granulomatous meningeal inflammation with associated bacterial foci, and histology of the spleen (120 h, Giemsa stain, 40×), showed large numbers of bacteria within degenerate splenic ellipsoids (FIG. 6B, Table 3). HSB challenged intraperitoneally with the S. iniae ΔPGM mutant similarly exhibited dissemination of the bacteria to blood, brain and spleen such that 4 hours post-infection bacterial counts in these tissues were only modestly less than those observed with WT infection (FIG. 6A). In stark contrast to the WT strain, the mutant was rapidly cleared from both blood and brain to undetectable levels within 24 h, and slowly cleared from the spleen to undetectable levels by 5 days post-infection (FIG. 6A). No mortalities were observed in the ΔPGM mutant-infected group of HSB. Histological examination of brain and spleen from fish infected with the ΔPGM mutant did not reveal evidence of the inflammatory damage produced in WT-infected HSB (FIG. 6B, Table 3).

TABLE 3
Histological survey of brain and spleen of hybrid striped bass infected
with wild-type Streptococcus iniae and the ΔPGM mutant

| | No. of samples infected/no. | | | | |
|---|---|---|---|---|---|
| | tested (h) | | | | |
| | | WT | | WT | |
| | | S. iniae | ΔPGM | S. iniae | ΔPGM |
| Test and tissue studied | PBS | (48) | (48) | (96) | (96) |
| Giemsa stain | | | | | |
| Spleen: bacteria | 0/2 | 3/3 | 0/3 | 3/3 | 0/3 |
| associated with ellipsoids | | | | | |
| Brain: bacteria associated | 0/2 | 3/3 | 0/3 | 3/3 | 0/3 |
| with meninges | | | | | |
| Brain histology | | | | | |
| Meningeal inflammation | 0/2 | 2/3 | 0/3 | 3/3 | 0/3 |
| Ventricle (optic lobe) | 0/2 | 0/3 | 0/3 | 2/2 | 0/3 |
| inflammation | | | | | |
| Relative severity of | | | | | |
| histological lesions (0-4) | | | | | |
| Spleen | 0 | 4 | 1 | 3 | 1 |
| Brain | 0 | 3 | 0 | 4 | 0 |

### EXAMPLE 6

### Vaccination Against S. iniae Infection with Live-Attenuated Vaccination with ΔPGM Mutant

### Vaccine Trial

To test the ability of TnM2 to function as a live-attenuated vaccine, groups of 40 fish (approximately 30 g) were injected IP with 4×10⁵, 4×10⁶, 4×10⁷ or 4×10⁸ cfu of TnM2 in PBS. Controls (40) were injected with PBS alone. Fish were held for 2,000 degree-days (approximately 4 months); no mortalities were observed during the holding period. Fish were then challenged by injection with the previously determined lethal dose (4×10⁵ cfu) of K288, held in mixed groups for 21 days at 24-27° C., and monitored for mortality. Brain biopsy cultures were taken from all mortalities and cultured to confirm S. iniae meningoencephalitis as the cause of death.

Results: Efficacy of Live-Attenuated Vaccination with ΔPGM Mutant Against S. iniae Infection.

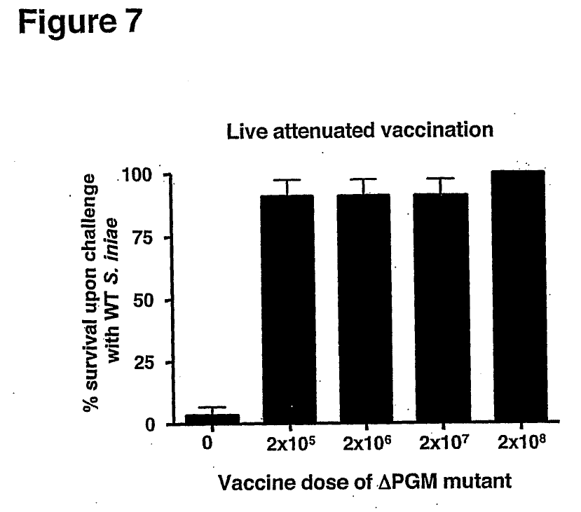

It was hypothesized that the aborted infectious process of the attenuated ΔPGM mutant could elicit an immune response capable of protecting HSB against subsequent WT S. iniae infection. A vaccination trial was performed by infecting HSB with various concentrations of the S. iniae ΔPGM mutant TnM2 (or PBS as a negative control), allowing them to spontaneously clear the infection, then housing them for a period of 2,000 degree-days (approximately 4 months). At this point, all animals were challenged with a typically lethal dose (4×10⁵ cfu) of WT S. iniae strain K288. Only 3% survival was observed in control fish that were mock immunized with PBS, while all groups of fish that were previously challenged with varying doses of the attenuated ΔPGM mutant experienced at least 90% survival (FIG. 7). At the highest concentration of mutant used for live-attenuated vaccination (2×10⁸ cfu), 100% of fish survived subsequent challenge with a lethal dose of the WT S. iniae strain. This trial indicates that the ΔPGM mutant TnM2 elicits an effective immune response and may have value as a live-attenuated vaccine to protect HSB against S. iniae infection.

### EXAMPLE 7

### Deletion and Replacement of the S. iniae PGM Gene by Homologous Recombination and Efficacy of PGM⁻ Mutants as Vaccines

Deletion and replacement mutations of the S. iniae pgm gene are generated by homologous recombination in vivo using plasmids constructed to contain terminal sequences from the pgm gene. Following introduction of the plasmid into S. iniae, sequences of the pgm gene in the S. iniae chromosome recombine with homologous sequences on the plasmid resulting in replacement (insertion) or deletion of the chromosomal pgm sequence between the terminal pgm sequences.

Briefly, double stranded oligonucleotide adaptors corresponding to the 5′ and 3′ ends of the S. iniae pgm gene (SEQ ID NO:5) are synthesized and ligated (in the same order and orientation as they are found on the bacterial chromosome) to the ends of a selectable antibiotic resistance marker (Cm^{R}). For insertion mutagenesis, the selectable marker is placed between S. iniae pgm adaptors. For deletion of pgm gene sequences without replacement, the selectable marker is positioned outside the 5′ and 3′ pgm sequences. The adaptors are designed with terminal restriction sites, permitting digestion with appropriate restriction enzymes for insertion into the multiple cloning site of plasmid pGBS1, which is a temperature sensitive vector suitable for direct transformation into Streptococcus strains. See Framson et al, supra. The adaptor-ligated Cm resistance marker is digested with appropriate restriction enzymes to generate cohesive termini and ligated into the multiple cloning site of pGBS1. The resulting pgm recombination vector is introduced into competent S. iniae cells by electroporation as described above in Example 1. Id.

Transformants are selected on appropriate antibiotic selective media. Cm^{R} colonies are expanded and assayed for PGM activity as described above in Example 3. Replacement or deletion of the pgm gene is confirmed in phosphoglucomutase-negative cultures by PCR.

In certain experiments, the recombinant plasmid is eliminated from the S. iniae following recombination by growing the cells at the non-permissive temperature for plasmid replication as described by Framson et al. See id. at 3544-45.

To test the ability of site-directed pgm deletion and insertion mutants of S. iniae to function as live-attenuated vaccines, groups of 40 fish (approximately 30 g) are injected IP with 4×10⁵, 4×10⁶, 4×10⁷ or 4×10⁸ cfu of mutant bacteria in PBS. Controls (40) are injected with PBS alone. Fish are held for 2,000 degree-days (approximately 4 months); no mortalities are observed during the holding period. Fish are then challenged by injection with the previously determined lethal dose (4×10⁵ cfu) of 88, held in mixed groups for 21 days at 24-27° C., and monitored for mortality. Brain biopsy cultures are taken from all mortalities and cultured to confirm S. iniae meningoencephalitis as the cause of death. Fish vaccinated with pgm⁻ S. iniae show reduced mortality when subsequently challenged with K288, thereby demonstrating the efficacy of pgm⁻ S. iniae as a live attenuated vaccine.

Although the invention has been described with reference to the above examples, it will be understood that modifications and variations are encompassed within the spirit and scope of the invention. Accordingly, the invention is limited only by the following claims.

What is claimed is:

1. An isolated Streptococcus iniae bacterium comprising a phosphoglucomutase deficiency.

2. The isolated bacterium of claim 1, wherein phosphoglucomutase enzyme activity is decreased at least about 10-fold compared to a wild type Streptococcus iniae bacterium.

3. The isolated bacterium of claim 1, wherein the bacterium is more sensitive to immune clearance mechanisms than a wild type Streptococcus iniae bacterium.

4. The isolated bacterium of claim 3, wherein sensitivity to immune clearance mechanisms comprises sensitivity to at least one antimicrobial peptide.

5. The isolated bacterium of claim 4, wherein the at least one antimicrobial peptide is moronecidin or mCRAMP.

6. The isolated bacterium of claim 1, wherein the bacterium comprises at least one phenotypic difference when compared to a wild type Streptococcus iniae bacterium selected from the group consisting of: decreased buoyancy; increased cell hydrophobicity; decreased surface associated exopolysaccharide capsule; decreased binding to cytochrome C; decreased surface negative charge; and increased cell volume.

7. The isolated bacterium of claim 1, wherein the bacterium comprises a mutation in a gene encoding a phosphoglucomutase.

8. The isolated bacterium of claim 7, wherein the mutation is an insertion or a deletion.

9. The isolated bacterium of claim 8, wherein the insertion comprises a transposon.

10. The isolated bacterium of claim 8, wherein the insertion comprises a selectable marker.

11. The isolated bacterium of claim 8, wherein the insertion or deletion is generated by site-directed mutagenesis.

12. The isolated bacterium of claim 7, wherein the mutation is in a promoter.

13. The isolated bacterium of claim 1, wherein the bacterium does not produce phosphoglucomutase protein.

14. The isolated bacterium of claim 1, wherein the bacterium produces avirulent phosphoglucomutase protein.

15. The isolated bacterium of claim 1, wherein the bacterium is avirulent in an aquatic species.

16. The isolated bacterium of claim 7, wherein the phosphoglucomutase comprises the amino acid sequence set forth in SEQ ID NO:6.

17. The bacterium of claim 7, wherein the phosphoglucomutase is encoded by a polynucleotide comprising the sequence set forth in SEQ ID NO:5.

18. A vaccine comprising the bacterium of claim 1.

19. The vaccine of claim 18, wherein the bacterium is live.

20. The vaccine of claim 18, further comprising at least one additional component selected from an adjuvant, a stabilizer and a diluent.

21. A pharmaceutical or veterinary composition comprising the bacterium of claim 1 and a suitable carrier.

22. A method for preventing Streptococcus iniae disease in a subject comprising administering the bacterium of claim 1 to a subject, wherein the subject develops immunity to the bacterium, thereby preventing Streptococcus iniae disease.

23. The method of claim 22, wherein the Streptococcus iniae disease is meningoencephalitis.

24. The method of claim 22, wherein the subject is a aquatic species.

25. The method of claim 24, wherein the aquatic species is a tilapia, a hybrid striped bass, a channel catfish, a rainbow trout, an eel, a yellowtail, a turbot or a sea bass.

26. The method of claim 24, wherein the aquatic species is a fish.

27. The method of claim 26, wherein the fish species is a tilapia or a hybrid striped bass. a channel catfish, a rainbow trout, an eel, a yellowtail, a turbot of a sea bass.

28. The method of claim 22, wherein the bacterium is administered intraperitoneally, subcutaneously, intravenously, intramuscularly, orally, or by immersion.

29. An isolated polynucleotide comprising the sequence set forth in SEQ ID NO:5.

30. A primer comprising 15-50 nucleotides of the sequence set forth in SEQ ID NO:5.

31. The isolated polynucleotide of claim 29, wherein the polynucleotide is contained in a plasmid.

32. The isolated polynucleotide of claim 31, wherein the plasmid is pSiPGM.

33. An isolated polypeptide comprising the sequence set forth in SEQ ID NO:6.

34. The isolated polypeptide of claim 33, wherein the polypeptide is expressed from an expression vector.

35. The isolated polypeptide of claim 34, wherein the expression vector is pSiPGM.

36. A method for identifying a virulence factor in a marine pathogen, comprising the steps of:
(a) randomly mutagenizing cells of a marine pathogen at the rate of one mutation per cell, thereby preparing a population of randomly mutated pathogens;
(b) isolating clones of the randomly mutated pathogens;
(c) identifying a mutant having reduced virulence by comparing the virulence of a clone of the randomly mutated pathogens with the unmutagenized pathogen; and
(d) determining the nucleotide position of the mutation in the mutant of reduced virulence, thereby identifying a virulence factor in a marine pathogen.

37. The method of claim 36, wherein the marine pathogen infects an aquatic species.

38. The method of claim 37, wherein the aquatic species is a tilapia or a hybrid striped bass.

39. The method of claim 36, wherein the marine pathogen is a bacterium.

40. The method of claim 39, wherein the bacterium is Streptococcus iniae.

41. The method of claim 36, wherein randomly mutagenizing comprises transposon-mediated mutagenesis.

42. The method of claim 41, wherein the transposon is Tn917.

43. The method of claim 36, wherein identifying a mutant of reduced virulence by comparing the virulence of clone of the randomly mutated pathogens with the unmutagenized pathogen comprises:
(i.) infecting a first subject susceptible to the marine pathogen with a clone of the randomly mutated pathogens;
(ii.) infecting a second subject susceptible to the marine pathogen with the unmutagenize marine pathogen; and
(iii.) comparing the pathogenic response of the first subject and the second subject, wherein a lesser pathogenic response of the first subject compared to the second subject is indicative of reduced virulence of the mutant,
thereby identifying a mutant of reduced virulence.

44. A transposon insertion library comprising a plurality of S. iniae bacteria prepared according to the method of claim 43.

45. An avirulent bacterium comprising a deficiency in a virulence gene, wherein the bacterium is one of the plurality of S. iniae bacteria of the library of claim 44.

46. The bacterium of claim 45, wherein the bacterium is avirulent in an aquatic species.

47. The avirulent bacterium of claim 46, wherein the virulence gene is ABC transporter, integrase, recombinase, transposase, tRNA synthetase, or membrane protein.


SEQUENCE LISTING (11 sequences)

SEQ ID NO: 1 - DNA, 26 bp, Artificial sequence
  Primer
gaactagctagttacttttgtaactg

SEQ ID NO: 2 - DNA, 26 bp, Artificial sequence
  Primer
ctaattcacaaaagtgttgatttcag

SEQ ID NO: 3 - DNA, 28 bp, Artificial sequence
  Primer
gaaacattggtttagtgggaatttgtac

SEQ ID NO: 4 - DNA, 146 bp, Streptococcus iniae
  misc_feature: Transposon
taattgttgaactagctagttacttttgtaactggctttttctgaaaattccaagctgaaaactcctgtaaatatctgtaaaaaaatgagtgattttttcttatgaaaatggtatataatgaaagcgtttaaaaatattaaggaga

SEQ ID NO: 5 - DNA, 1716 bp, Streptococcus iniae
atgacttatacagaaaattatcaaaaatggcttaatgcctcaaacctccctgattatctaaaagatgaacttcttgcaatggatgaaaaaacaaaggaagatgctttctacacaaacttagagtttggaactgcaggtatgcgtggttatattggtgcaggaacaaatcgcattaatgtttttgttgttcgtcaagctactgaagggttagcaaaattagtagaatcaaaagggcaagcagccaaagatgctggtgttgctattgcatacgattcccgccacttctcacctgaatttgcatttgaatcagcacaagtattagctgctcacggcatcaaatcatatgtctttgaaagcttgcgccctacaccagaattatcttttgcggttcgccatttaggtgccgttgcgggcattatggtaacagcaagtcataatccagctccatttaatggctacaaagtttatggtgctgatggcggccaaatgcctcctgcagacgcagacgccctcacagactacattagagctattgaagatccattttctattgtattagctgacttagaagactcaaaatctaatggtctcattgaagttattggggaggctattgacacagaataccttaaagaagttaaagatgttactatcaatcaagaacttattgatacctttggccgtgatatgaaaattgtctacacaccattacatggtactggtgaaatgttgacgcgtcgcgcacttgcacaagctggctttgagtcagttgaagttgttgaaagtcaagccaaagcagaccctaatttctcaactgtagcatctcctaatccagaaagtcaagaagcatttgctttagcagaagaattaggaagaacagttaatgcagatgttttggttgcgactgaccctgatgccgatcgtcttggagttgaaattcgtcaagctgatggttcttattggaacttatctggtaaccaaattggtgcccttattgctaaatatatcttggaagcacataaagtagcaggaaccctaccagtcaatgctgcacttgcaaaatcaatcgtgtctactgaattggtaacaaaaattgctgagagttatggcgcaaccatgtttaatgycttaacaggctttaaatttattgcagaaaaaattcaagaatttgaagaaacacataaccatacctatatgtttggatttgaagaaagttttggttacctcattaaaccttttgtacgtgacaaagatgctattcaagcggtacttattgttgctgaaattgcagcttactaccgctctaaaggattaacacttgcagatggtattgatgacatttataaagaatatggttactttgcagaaaaaaccatttcattgacactttctggtgttgacggtgcaagtcaaatcaaagctatcatggacaaattccgtcaaagtgctcctactagcttcaatcaaacagaaattgttctatcagaagacttcttagaacaaactgctaaatctgctgatggcacaagtccattaacaacaccgccaagcaatgtcttaaaatacacacttgctgatgattcttggtttgctgttagaccatccggaacagaaccgaaaatcaaattctatgttgctactgttggccaaacacttttagaagctgaagaaaaaatcaaaacaattgaagctgaaatcaacacttttgtgaattag

SEQ ID NO: 6 - protein, 571 aa, Streptococcus iniae
  MISC_FEATURE (376)..(376): X is Val or Ala
MTYTENYQKWLNASNLPDYLKDELLAMDEKTKEDAFYTNLEFGTAGMRGYIGAGTNRINVFVVRQATEGLAKLVESKGQAAKDAGVAIAYDSRHFSPEFAFESAQVLAAHGIKSYVFESLRPTPELSFAVRHLGAVAGIMVTASHNPAPFNGYKVYGADGGQMPPADADALTDYIRAIEDPFSIVLADLEDSKSNGLIEVIGEAIDTEYLKEVKDVTINQELIDTFGRDMKIVYTPLHGTGEMLTRRALAQAGFESVEVVESQAKADPNFSTVASPNPESQEAFALAEELGRTVNADVLVATDPDADRLGVEIRQADGSYWNLSGNQIGALIAKYILEAHKVAGTLPVNAALAKSIVSTELVTKIAESYGATMFNXLTGFKFIAEKIQEFEETHNHTYMFGFEESFGYLIKPFVRDKDAIQAVLIVAEIAAYYRSKGLTLADGIDDIYKEYGYFAEKTISLTLSGVDGASQIKAIMDKFRQSAPTSFNQTEIVLSEDFLEQTAKSADGTSPLTTPPSNVLKYTLADDSWFAVRPSGTEPKIKFYVATVGQTLLEAEEKIKTIEAEINTFVN

SEQ ID NO: 7 - protein, 572 aa, Streptococcus pneumoniae
MSYQENYQKWVDFVELPDYLRQDLENMDEKTKEDAFYTNLEFGTAGMRGLVGAGTNRINIYVVRQATEGLARLIESKGGNEKERGVAIAYDSRHFSPEFAFESAAVLAKHGIKSYVFESLRPTPELSFAVRHLNCFAGIMVTASHNPAPFNGYKVYGEDGGQMPPHDADALTTYIRAIENPFAVEVADVETEKASGLIEVIGEAVDVEYLKEVKDVNINPALIEEFGKDMKIVYTPLHGTGEMLARRALAQAGFDSVQVVEAQATADPDFSTVTSPNPESQAAFALAEELGRQVGADVLVATDPDADRVGVEVLQKDGSYLNLSGNQIGAIMAKYILEAHKNAGTLPENAALCKSIVSTDLVTKIAESYGATMFNVLTGFKFIAEKIQEFEEKHNHTYMMGFEESFGYLIKPFVRDKDAIQAVLVVAELAAYYRSRGLTLADGIEEIYKEYGYYAEKTISVTLSGVDGAEQIKAIMAKFRNNAPKEWNETAITVVEDFKAQTATVADGTVTNLTTPPSDVLKYTLADSSWIAVRPSGTEPKIKFYIAVVGETNEESQAKIANIEAEINAFVK

SEQ ID NO: 8 - protein, 572 aa, Streptococcus thermophilus
MSYTENYQKWLDFAELPAYLRDELVSMDEKTKEDAFYTNLEFGTAGMRGLIGAGTNRINIYVVRQATEGLAQLIDSKGEEAKKRGVAIAYDSRHFSPEFAFESAQVLAAHGIKSYVFESLRPTPELSFAVRHLHTFAGIMITASHNPAPFNGYKVYGEDGGQMPPADADALTDYIRAIDNPFTVKLADLEDSKASGLIEIIGENVDAEYLKEVKDVNINQDLINEYGRDMKIVYTSLHGTGEMLVRRALAQAGFDAVQVVEAQAVPHADFLTVKSPNPENQDAFALAEELGRNVDADVLVATDPDADRLGVEIRQPDGSYLNLSGNQIGAIIAKYILEAHKTAGTLPANAALCKSIVSTELVTKIAESYGATMFNVLTGFKFIGEKIHEFETQHNYTYMFGFEESFGYLIKPFVRDKDAIQAVLIVAEIAAYYRSRGMTLADGIEEIYKQYGYFSEKTISVTLSGVDGAAEIKKIMDKFRRNAPKQFNNTDIAKTEDFLEQTATTADGVEKLTTPPSNVLKYILADDSWFAVRPSGTEPKIKFYIATVGETEADAKEKIANIEAEINAFVGE

SEQ ID NO: 9 - protein, 581 aa, Bacillus subtilis
MTWRKSYERWKQTEHLDLELKERLIELEGDEQALEDCFYKDLEFGTGGMRGEIGAGTNRMNIYTVRKASAGFAAYISKQGEEAKKRGVVIAYDSRHKSPEFAMEAAKTLATQGIQTYVFDELRPTPELSFAVRQLNAYGGVVVTASHNPPEYNGYKVYGDDGGQLPPKEADIVIEQVNAIENELTITVDEENKLKEKGLIKIIGEDIDKVYTEKLTSISVHPELSEEVDVKVVFTPLHGTANKPVRRGLEALGYKNVTVVKEQELPDSNFSTVTSPNPEEHAAFEYAIKLGEEQNADILIATDPDADRLGIAVKNDQGKYTVLTGNQTGALLLHYLLSEKKKQGILPDNGVVLKTIVTSEIGRAVASSFGLDTIDTLTGFKFIGEKIKEYEASGQYTFQFGYEESYGYLIGDFARDKDAIQAALLAVEVCAFYKKQGMSLYEALINLFNEYGFYREGLKSLTLKGKQGAEQIEAILASFRQNPPQKMAGKQVVTAEDYAVSKRTLLTESKEEAIDLPKSNVLKYFLEDGSWFCLRPSGTEPKVKFYFAVKGSSLEDSEKRLAVLSEDVMKTVDEIVESTAK

SEQ ID NO: 10 - protein, 545 aa, Escherichia coli
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAISNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKN

SEQ ID NO: 11 - DNA, 709 bp, Streptococcus iniae
caaattcctcgtaggcgctcgggaccccaatggcgaagattttaagaatttaaacgcaaaagtgtggaagaacaagtagcttatgtccctcaagagcatatcttgttttctaaaactgtttctgaaaatattgcttttggaactaaaaatccaagttaccaagatattgcagaagcgattgctacagcggatttcactaaagatttaaaacatatgccaaaaggtttagagacccaaattggagaaagaggattgtctatctcaggaggtcaaaaacaacgggtatctattgcaagagcctttttggccaagtctgatttattaattttagatgattccttatctgctgtggatgctaaaacagaaacaaaaattatcgcaaatattcaaaaggttaggaaaaataaaacgacatttatcgtcagccaccgattatctgctgttcaacatgcagattgggttatcgttttagagcaggggcgtatcagcgaagaaggcacccctgatgaccttttaaagaaaaaaggttggtattatgaacaatatcaacggcaacaagcacaggaggactaaaatgtccctatttataaaattaagtaaagagttaaaagttgctaaaataagttttatctcaggattattactcttattagtagcttcagccctggggcaagtttcccccatattgatgaagcagatgattgatcat